(19) United States
(12) Patent Application Publication
(10) Pub. No.: US 2025/0271447 A1
(43) Pub. Date: Aug. 28, 2025

(54) MULTIPLEXING HOMOCYSTEINE AND CYSTEINE IN FIRST-TIER SCREENING ASSAYS

(71) Applicant: THE UNITED STATES OF AMERICA, AS REPRESENTED BY THE SECRETARY, DEPARTMENT OF HEALTH AND HUMAN SERV, Bethesda, MD (US)

(72) Inventors: Charles Austin Pickens, Stone Mountain, GA (US); Konstantinos Petritis, Peachtree Corners, GA (US)

(21) Appl. No.: 18/858,464

(22) PCT Filed: Apr. 19, 2023
(86) PCT No.: PCT/US2023/019116

§ 371 Date: Oct. 21, 2024

Related U.S. Application Data
(60) Provisional application No. 63/333,647, filed on Apr. 22, 2022.

(51) Int. Cl. G01N 33/68; G01N 30/06; G01N 30/88

(52) U.S. Cl. CPC G01N 33/6848; G01N 30/06; G01N 30/88; G01N 33/6815; G01N 2030/067; G01N 2030/8818; G01N 2030/8822; G01N 2800/7076

(57) ABSTRACT

A method of multiplexing Hcy and/or Cys in a first-tier screening assay includes: contacting a blood sample with a reducing agent optionally in the presence of a solvent to convert at least one of a Hcy dimer, a Hcy-protein complex, a Cys dimer different from the homocysteine dimer, or a Cys-protein complex in the blood sample to a Hcy and/or Cys monomer thereby forming a monomer sample; reacting the Hcy and/or Cys monomer in the monomer sample with a thiol derivatizing agent to form a thiol derivatized sample comprising a thiol derivatized Hcy and/or Cys monomer; and optionally converting a carboxylic acid or a carboxylate group in the thiol derivatized Hcy and/or Cys monomer to an ester, forming a thiol-and-ester derivatized sample comprising a thiol-and-ester derivatized Hcy and/or Cys monomer. The method can be used to determine a level of tHcy, a level of tCys, and/or a level of CysT in a blood sample, and screen for CBS deficiency, HCU, or hyperhomocysteinemia.

### CROSS-REFERENCE TO RELATED APPLICATION

This application claims priority of U.S. Provisional Application No. 63/333,647, filed Apr. 22, 2022, the content of which is incorporated herein by reference in its entirety.

### STATEMENT OF GOVERNMENT SUPPORT

This invention was made in part with government support from the US Department of Health and Human Services, National Institutes of Health. The Government has certain rights in this invention.

### BACKGROUND OF THE INVENTION

### 1. Field of the Invention

This invention relates generally to methods that allow first-tier screening of homocysteine in newborns.

### 2. Brief Description of the Art

Each year, millions of babies are routinely screened using a few drops of blood from the newborn's heel to identify presymptomatic newborns at risk for disorders such as inborn errors of metabolism (IEM). IEM often results in perturbed levels of amino acids or acylcarnitines, which can be used as diagnostic biomarkers for IEM. Most IEM biomarkers can be detected by a primary-screen flow injection analysis (FIA) coupled to tandem mass spectrometry (MS/MS) analysis. Commonly referred to as first-tier or primary-tier screening, a multiplexed FIA-MS/MS assay can be used to analyze from one to more than 45 biomarkers with a sub two-minute analysis time.

Homocystinuria (HCU) is an IEM often caused by deficiency most commonly in the enzyme cystathionine b-synthase (CBS). CBS deficiency can lead to elevated concentrations of blood/plasma and urine homocysteine (Hcy) as well as increased concentration of blood/plasma methionine. Clinical symptoms in untreated or late treated patients include Marfan-like appearance, progressive myopia and lens dislocation, thromboembolism, epilepsy, osteoporosis, neurological disorders, etc. Early detection and treatment is critical, and can prevent CSB deficiency-associated morbidities. Therefore, HCU newborn screening is a routine test recommended by the Advisory Committee on Heritable Disorders in Newborns and Children.

Due to the complex chemistry of Hey, along with known and unknown interferences, labs are unable to screen for this biomarker in first-tier newborn screening assays. Instead, labs use methionine as a surrogate marker for HCU. However, methionine concentrations physiologically vary in newborn blood samples which can impact false positive and false negative rates, and reflex rates to second-tier screening. Furthermore, methionine can take several days to reach abnormal levels in newborns with HCU while specimen collection is recommended at 24-48 h contributing to false negatives. In fact, stakeholders currently estimate that nearly 50% of HCU cases are missed due to the use of methionine as the biomarker in first-tier HCU screening.

One way to address the issue is by using a second-tier screening assay following the first-tier screening assay to test for total Hcy (tHcy) in specimens that are presumptive positives due to a methionine elevation. Second-tier screening tests employ a separation step that separates Hcy from other compounds in the matrix before detection by tandem mass spectrometry analysis. This approach has several disadvantages such as that specimens may not exceed the methionine cutoff in order to be reflexed for second-tier screening, additional costs with maintaining a separate assay and capital equipment required, as well as added time before referral.

Recently, an assay has been published by Pickens et al. in Clinical Chemistry, Volume 67, Issue 12, Pages 1709-1720, where first- and second-tier screening analytes (including Hcy) can be multiplexed in one assay using capillary electrophoresis. That approach has several limitations associated with the use of the on-chip electrophoresis device and is lower throughput. Thus, low-cost, first-tier screening assay for Hcy is needed for effectively assessing risk factors for various diseases.

### SUMMARY OF THE INVENTION

A method of multiplexing Hcy, cysteine (Cys), or a combination thereof in a first-tier screening assay includes: contacting a blood sample with a reducing agent optionally in the presence of a solvent to convert at least one of a Hcy dimer, a Hcy-protein complex, a Cys dimer different from the Hcy dimer, or a Cys-protein complex in the blood sample to a Hcy monomer, a Cys monomer, or a combination thereof thereby forming a monomer sample; reacting the Hcy monomer, the Cys monomer, or a combination thereof in the monomer sample with a thiol derivatizing agent to form a thiol derivatized sample comprising a thiol derivatized Hcy monomer, a thiol derivatized Cys monomer, or a combination thereof; and optionally converting a carboxylic acid or a carboxylate group in the thiol derivatized Hcy monomer, the thiol derivatized Cys monomer, or a combination thereof to an ester, preferably a butyl ester forming a thiol-and-ester derivatized sample comprising a thiol-and-ester derivatized Hcy monomer, a thiol-and-ester derivatized Cys monomer, or a combination thereof.

A method of determining a level of tHcy, a level of total cysteine (tCys), or a combination thereof in a blood sample includes: multiplexing Hcy, Cys, or a combination thereof in a first-tier screening assay according to a method as described herein, and analyzing the thiol derivatized sample, the thiol-and-ester derivatized sample, or a ready-to-use sample with mass spectrometry to quantify a level of tHcy, a level tCys, or a combination thereof in the blood sample.

A method of screening for CBS deficiency, HCU, or hyperhomocysteinemia includes: providing a blood sample from a subject; determining a level of tHcy in the blood sample according to a method as described herein; and determining that the subject has CBS deficiency, HCU, or hyperhomocysteinemia if the level of tHcy in the blood sample is above a threshold.

### BRIEF DESCRIPTION OF THE DRAWINGS

A description of the figures, which are meant to be exemplary and not limiting, is provided in which:

FIG. 1 illustrates an embodiment of a method of multiplexing Hcy, Cys, or a combination thereof in a first-tier screening assay;

FIG. 2 illustrates another embodiment of a method of multiplexing Hcy, Cys, or a combination thereof in a first-tier screening assay;

FIG. 3 illustrates yet another embodiment of a method of multiplexing Hcy, Cys, or a combination thereof in a first-tier screening assay;

FIG. 4 shows the thiol derivation of a Hcy monomer with N-ethyl maleimide (Hcy-NEM);

FIG. 5 shows the possible fragmentation of Hcy-NEM during a mass spectrometry analysis;

FIG. 6 shows a mass spectrum of Hcy-NEM;

FIG. 7 shows a high resolution mass spectrum of Cys-NEM;

FIG. 8 shows a high resolution mass spectrum of Cystathionine (CysT);

FIG. 9 compares the analysis of DBS extracts with or without TCEP and NEM treatment, where FIG. 9A and FIG. 9B depict the analysis results of Hcy (A) and Met-D3 (B) from DBS extracts in the absence of TCEP and NEM using a hydrophilic interaction chromatography (HILIC) pre-column coupled to MS/MS; and FIG. 9C and FIG. 9D display the analysis results of DBS extracts treated with TCEP and NEM;

FIG. 10A and FIG. 10B compare the tHcy concentrations determined via FIA-MS and LC-MS in low quality control (QC) samples for non-butyl esters or butyl esters respectively; FIG. 10C and FIG. 10D compare the tHcy concentrations determined via FIA-MS and LC-MS in high QC samples for non-butyl esters or butyl esters respectively;

FIG. 11A shows the impacts of Hcy workflow using low QCs and non-butyl esters on amino acids/acylcarnitines (AA/AC) biomarkers; FIG. 11B shows the impacts of Hcy workflow using low QCs and butyl esters on AA/AC biomarkers; FIG. 11C shows the impacts of Hcy workflow using high QCs and non-butyl esters on AA/AC biomarkers; and FIG. 11D shows the impacts of Hcy workflow using high QCs and butyl esters on AA/AC biomarkers;

FIG. 12 compares the tHcy level of two proficiency test samples, one engineered to resemble a HCU positive specimen and another engineered to resemble a presumptive normal specimen using the method disclosed herein;

FIG. 13 displays the confirmation of DiNEM-DTT formation, where 13A shows the structures for NEM and DTT, the reaction product DiNEM-DTT and theoretical m/z and isotopic distribution, and the theoretical dissociation products of DiNEM-DTT; 13B shows high resolution mass spectrum obtained from a sample where NEM and DTT were allowed to react; and 13C shows the high resolution parallel reaction monitoring spectra of parent m/z for DiNEM-DTT, obtained from a sample where NEM and DTT were allowed to react; and

FIG. 14 displays a biplot of methionine and tHcy with developmental second-tier proficiency testing materials engineered as healthy and presumptive positive for HCU, along with three clinical NBS specimens including a healthy, a newborn that had total parenteral nutrition (TPN) administered, and a confirmed HCU positive case.

The above described and other features are exemplified by the following detailed description and Examples.

### DETAILED DESCRIPTION OF THE INVENTION

As used herein, the blood sample can be a dried blood spot sample, a serum sample, or a plasma sample, preferably the blood sample is a dried blood spot sample from a neonate.

First-tier assay refers to an assay that can analyze multiple biomarkers at the same time.

Second-tier assay refers to an assay that targets a specific biomarker which cannot be reliably screened in the first-tier assay due to the presence of known or unknown interferences.

Analysis of tHcy in a blood sample such as a dried blood spot by mass spectrometry, without prior chromatographic separation, results in poor quantitative accuracy due to endogenous and exogenous interferences. The inventors hereof have developed a method to selectively derivatize Hcy in this complex sample matrix using a thiol derivatizing agent such as N-ethyl maleimide, which allows accurate quantitation by shifting Hcy's mass and mass-to-charge ratio, thus removing interferences. The method can also be used to derivatize Cys using the thiol derivatizing agent. The technology offers improved high-throughput method of multiplexing tHcy, tCys, or a combination thereof with amino acids, acylcarnitincs, succinylacetone, adenosine, deoxyadenosine, guanidinoacetic acid, creatine, creatinine, and lysophospholipids in a first-tier screening assay, with higher HCU screening precision, without affecting the stability of the co-analyzed biomarkers. CysT, which does not require derivatization, can be quantified concomitantly with the tHcy and tCys using the method.

Hcy exists in different forms in blood. In addition to a Hcy monomer, a blood sample can contain at least one of a Hcy dimer or a Hcy-protein complex. As used herein, a Hcy-protein complex refers to a Hcy monomer bound to a protein. In a Hcy dimer, Hcy can be linked in disulfide bonds to various other thiols. A Hcy dimer can include a Hcy-Cys dimer or a Hcy-compound 1 dimer where an example of compound 1 is glutathione.

Cys also exists in different forms in blood. In addition to a Cys monomer, a blood sample can contain at least one of a Cys dimer or a Cys-protein complex. As used herein, a Cys-protein complex refers to a Cys monomer bound to a protein. In a Cys dimer, Cys can be linked in disulfide bonds to various other thiols. A Cys dimer can include a Cys-Hcy dimer (same as Hcy-Cys dimer) or a Cys-compound 2 dimer, wherein an example of compound 2 is glutathione.

Methods of multiplexing Hcy, Cys, or a combination thereof in a first-tier screening assay arc illustrated in FIG. 1 to FIG. 3. As used herein, multiplexing Hcy, Cys, or a combination thereof in a first-tier screening assay means that tHcy and/or tCys can be analyzed simultaneously with other analytes using one sample.

As shown in FIG. 1 to FIG. 3, a method of multiplexing Hcy, Cys, or a combination thereof in a first-tier screening assay comprises converting at least one of the Hcy dimer, the Hcy-protein complex, the Cys dimer different from the Hcy dimer, or the Cys-protein complex in blood sample 1 to a Hcy monomer, a Cys monomer, or a combination thereof using a reducing agent optionally in the presence of a solvent to form a monomer sample 2.

The solvent can comprise at least one of acetonitrile, methanol, or water. Other organic solvents can also be used as long as the solvents do not adversely affect the formation of the monomer sample. The solvent can further comprise an additive that can precipitate proteins, for example an acid. If desired, more than one solvent or solvent system can be used in a sequential manner. As a specific example, the blood sample can be treated with a first solvent containing a lower concentration of acetonitrile, followed by a second solvent containing more acetonitrile, or methanol or an additive to precipitate the protein. When used, a volume ratio of the acetonitrile relative to water in the solvent can be 95:5 to 20:80 or 90:10 to 30:70, preferably 90:10 to 70:30, and more preferably 85:15 to 75:25. Hcy methods known in the literature typically use more aqueous extraction solutions, which causes MS components to become dirtier especially under FIA conditions. The methods described herein utilize lower proportions of water and allow for faster sample drying and cleaner mass spectrometer source and other components. Depending on the specific acid used, the acid can be present in an amount of 0.01 to 5 volume percent, 0.01 to 1 volume percent, 0.01 to 0.5 volume percent or 0.01 to 0.1 volume percent based on a total volume of the solvent. Examples of the acid include formic acid, acetic acid, trifluoroacetic acid, oxalic acid, and sulfosalicylic acid. The acid can be used alone or in a combination of two or more acids. Other suitable organic acids may also be used.

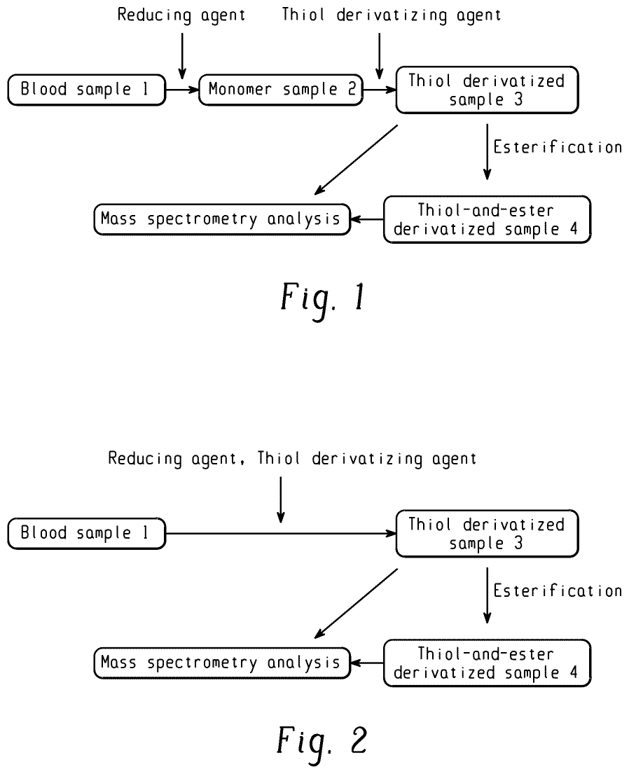

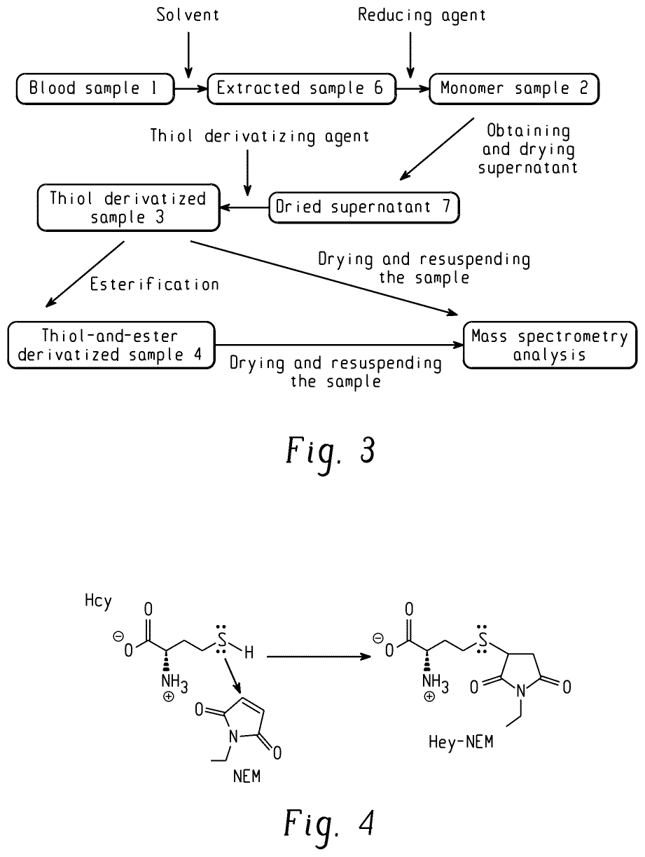

The reducing agent and the thiol derivatizing agent can be simultaneously or sequentially added to the blood sample 1. As shown in FIG. 1 and FIG. 3, the thiol derivatizing agent can be added after the monomer sample is formed. Alternatively, as shown in FIG. 2, the thiol derivatizing agent can be added before the monomer sample is formed.

The reducing agent comprises at least one of tris(2-carboxyethyl) phosphine (TCEP) or dithiothreitol (DTT). Preferably the reducing agent comprises TCEP. The reducing agent can be introduced as a solid or in a solution. The reducing agent can also be immobilized on a solid support such as nanoparticles, magnetic particles, and the like. As an example, the reducing agent is immobilized on magnetic particles, preferably magnetic nanoparticles, which allows removal of the reducing agent after reduction using a magnet, and the removal of the reducing agent in turn reduces potential ion suppression. As a specific example, TCEP immobilized on carbon coated cobalt nanoparticles can be used.

In an embodiment, the monomer sample 2 is prepared by mixing the blood sample 1 with a solvent, optionally at a temperature of 30° C. to 80° C., preferably 35° C. to 70° C., and more preferably 40° C. to 50° C. for 10 to 120 minutes, preferably 20 to 100 minutes, or more preferably 20 to 60 minutes to form an extracted sample 6; and contacting the extracted sample 6 with a reducing agent such as TCEP for 1 to 10 minutes to form the monomer sample 2. Preferably a concentration of TCEP is 10 to 50 millimolar (mM), 20 to 40 mM, or about 30 mM. The amount of the TCEP added to the extracted sample 6 can be 2 to 25 microliters (μL), 5 to 20 μL, or 8 to 18 μL, depending on the amount of the blood sample and the concentration of the reducing agent used.

Alternatively, the monomer sample 2 can be prepared by heating a blood sample 1 and a reducing agent such as DTT and TCEP in the presence of a solvent as described herein at a temperature of 30° C. to 80° C., 35° C. to 70° C., or 40° C. to 50° C. for 10 to 120 minutes, 20 to 100 minutes, or 20 to 60 minutes. Preferably a concentration of DTT or TCEP is 50 to 150 mM, 80 to 120 mM, or about 100 mM. The amount of the DTT or TCEP added can be 2 to 25 μL, 5 to 20 μL, or 8 to 18 μL, depending on the amount of the blood sample and the concentration of the reducing agent used.

The monomer sample can further contain a known amount of an internal standard. The internal standard can comprise at least one of an internal standard for an amino acid other than Hcy or Cys, an internal standard for an acylcarnitine, an internal standard for succinylacetone, or an internal standard for Hcy such as dHcy-²H₈ or dHcy isotopologues or isotopomers labeled with ¹³C, ¹⁵N, ¹⁸O, and/or ²H, or an internal standard for Cys such as dCys-¹³C₆²H₆¹⁵N₂ or dCys isotopologues or isotopomers labeled with ¹³C, ¹⁵N, ¹⁸O, and/or ²H. The internal standards are known and can comprise a stable isotope-labeled amino acid other than Hcy or Cys, a stable isotope-labeled acylcarnitine, a stable isotope-labeled succinylacetone, a stable isotope-labeled adenosine, a stable isotope-labeled deoxyadenosine, a stable isotope-labeled guanidinoacetic acid, a stable isotope-labeled creatine, a stable isotope-labeled creatinine, a stable isotope-label lysophospholipids, an internal standard for Hcy as described herein, or an internal standard for Cys as described herein. Other analyte internal standards can also be used. The internal standard can be separately added to the monomer sample, or can be added as part of the solvent added to the blood sample. In an embodiment, the internal standards are those in a working internal standard solution for first-tier screening containing dHcy-²H₈. Hcy interferences may or may not be present in the internal standard solution or the sample that being analyzed in the first-tier screening assay.

The monomer sample 2 can include a solid precipitate and a supernatant. Preferably, the supernatant is separated from the solid precipitate and optionally dried to form a dried supernatant 7.

The Hcy monomer, the Cys monomer, or a combination thereof in the monomer sample 2 or in the optionally dried supernatant 7 can be selectively derivatized with a thiol derivatizing agent to form a thiol derivatized sample 3. The derivatizing agent comprises an N-alkyl maleimide, and preferably the derivatizing agent comprises N-ethylmaleimide.

In an embodiment, 10 to 80 μL, 20 to 70 μL, or 30 to 50 μL of the thiol derivatizing agent can be added to blood sample 1, monomer sample 2, or optionally dried supernatant 7. A concentration of the derivatizing agent can be 10 to 80 mM, 20 to 60 mM, or 30 to 50 mM. The thiol derivatizing agent can be added as a solution in water and/or methanol. In an embodiment, the thiol derivatizing agent is added as a solution in water and method where water and methanol have a volume ratio of about 7:3 to about 3:7 or about 6:4 to about 4:6. Once added, the mixture of the Hcy monomer, the Cys monomer, or a combination thereof and the thiol derivatizing agent or the mixture of the blood sample with the reducing agent, and the thiol derivatizing agent can be shaken for 1 to 10 minutes or 2 to 8 minutes to form the thiol derivatized sample 3 comprising a thiol derivatized Hcy monomer, a thiol derivatized Cys monomer, or a combination thereof.

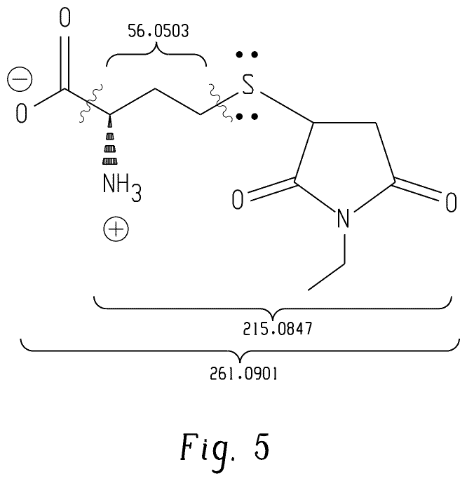

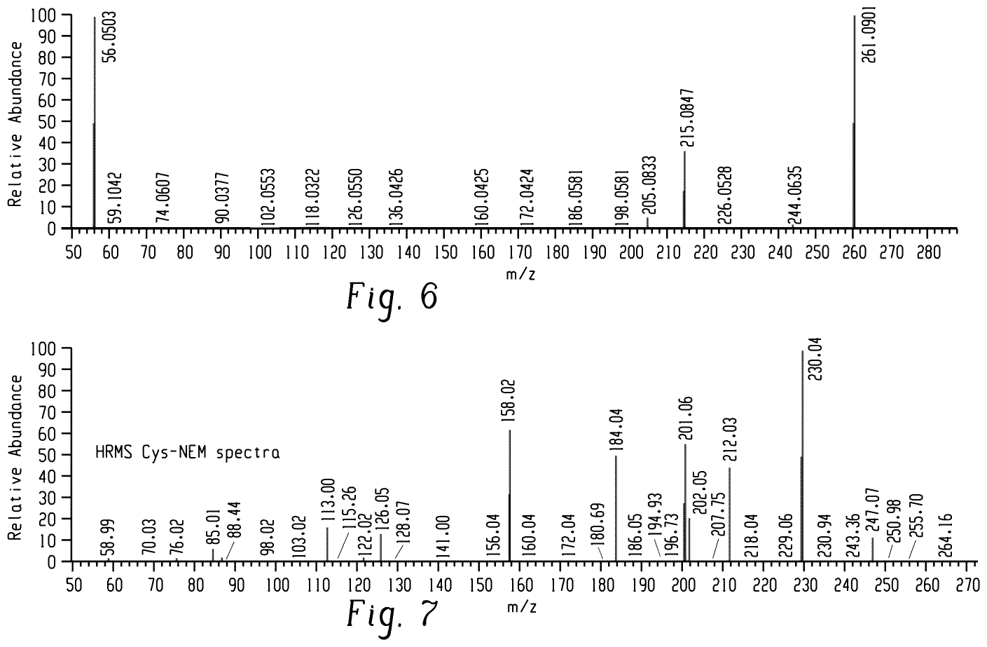

FIG. 4 shows the thiol derivation of a Hcy monomer with N-ethyl maleimide (Hcy-NEM). The possible fragmentation of Hcy-NEM during a mass spectrometry analysis is shown in FIG. 5, and a mass spectrum of Hcy-NEM is shown in FIG. 6. The selective derivation shifts Hcy's mass and mass-to-charge ratio, thus removing interferences and allowing for accurate quantitation of tHcy. Cys can also be modified with NEM forming Cys-NEM, simultaneously or separately with the derivation of Hcy. A mass spectrum of Cys-NEM is shown in FIG. 7. The data suggests the Cys-NEM transition 247.1>184.04 can perform well as the quantitative transition for Cys-NEM.

In a preferred embodiment, TCEP is used as the reducing agent, and NEM is used as the thiol derivatizing agent. The use of TCEP and NEM have a synergetic effect on the tHcy and/or tCys quantification, yielding higher signal which increases the sensitivity of the tHcy and/or tCys.

If desired, a carboxylic acid or a carboxylate group in the derivatized Hcy monomer, the derivatized Cys monomer, or a combination thereof can be converted to an ester, preferably a butyl ester forming a thiol-and-ester derivatized sample 4. Methods of forming an ester are not particularly limited. For example, the thiol derivatized Hcy monomer, the thiol derivatized Cys monomer, or a combination thereof can be heated with an alcohol such as a butanol in the presence of an acid catalyst to form the thiol-and-ester derivatized sample. As a specific example, 10 to 100 μL of butanol and an acid such as HCl are added to the thiol derivatized Hcy monomer, the thiol derivatized Cys monomer, or a combination thereof, and the mixture is placed in a heated oven at 40 to 80° C. for 5 to 60 minutes or 10 to 30 minutes to form the thiol-and-ester derivatized sample. The alcohol and the acid can be separately added to thiol derivatized Hcy monomer, the thiol derivatized Cys monomer, or a combination thereof, or the alcohol and the acid can be combined first before being added to the thiol derivatized Hcy monomer, the thiol derivatized Cys monomer, or a combination thereof.

Optionally the thiol derivatized sample 3 or the thiol-and-ester derivatized sample 4 can be dried to form a dried derivatized sample; and then a second solvent is added to the dried derivatized sample to resuspend the thiol derivatized Hcy and/or Cys monomer or the thiol-and-ester derivatized Hcy and/or Cys monomer, forming a ready-to-use sample. The second solvent can be an organic solvent and water mixture. Specific examples of the second solvent include acetonitrile and water mixture and methanol and water mixture. The volume ratio of the organic solvent relative to water in the solvent can be 95:5 to 20:80 or 90:10 to 30:70, preferably 90:10 to 70:30, and more preferably 85:15 to 75:25. The second solvent can also comprise an acid such as formic acid, acetic acid, trifluoroacetic acid, oxalic acid, and sulfosalicylic acid. The acid can be used alone or in a combination of two or more acids. Other suitable organic acids may also be used. Depending on the specific acid used, the acid can be present in an amount of 0.01 to 5 volume percent, 0.01 to 1 volume percent, 0.01 to 0.5 volume percent or 0.01 to 0.1 volume percent based on a total volume of the second solvent. The drying and the resuspending steps can be omitted, and the thiol derivatized sample 3 or the thiol-and-ester derivatized sample 4 can be used directly for analysis.

The thiol derivatized sample, the thiol-and-ester derivatized sample, or the ready-to-use sample can be analyzed with mass spectrometry with or without prior chromatography separation to quantify a level of tHcy, a level of tCys, or a combination thereof in the blood sample. As used herein, without prior chromatography separation includes the instance where a sample is directly injected into a mass spectrometer as well as the instance where a sample is injected to a column in “pass-through” mode (no chromatographic separation) before being introduced to a mass spectrometer. If chromatography separation is desired, pre-columns (often referred to as guard columns), analytical chromatography columns, and pre-columns coupled to analytical chromatography columns can be used. Preferably, these columns are used online, where a solvent and a sample to be analyzed can first pass through a column then flow continuously to a mass spectrometer. In preferred embodiments, when liquid chromatography is used, for the sake of throughput which is important for a screening method, the technique used is ultra or high performance liquid chromatography where the mobile phase is forced through a packed column with dimensions typically 1-50 mm in length, internal diameter of 5 mm or less and particle diameter of 10 μm or less. The chromatography column can separate isomers that may be difficult to differentiate by mass spectrometry and/or limit the ion-suppression by introducing simple mixtures of analytes at one time. In an embodiment the thiol derivatized sample, the thiol-and-ester derivatized sample, or the ready-to-use sample is analyzed with tandem mass spectrometry, and preferably a flow injection analysis, direct injection, or acoustic injection coupled to tandem mass spectrometry. With or without chromatographic separation, the total analysis time per sample is preferably 3 minutes or less. As used herein, the total analysis time means the total amount of time that it takes to obtain the analysis results (for example a level of tHcy and/or a level of tCys) after a sample is injected into a mass spectrometer (if no prior column is used) or after the sample is injected into a column before being introduced to a mass spectrometer.

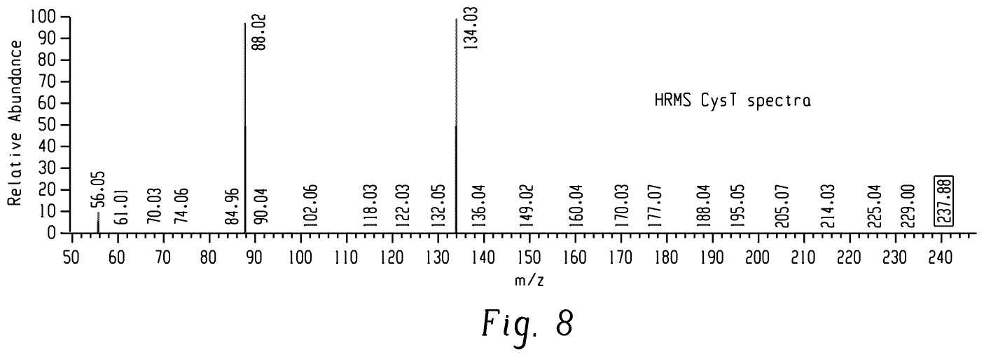

CysT, which is an intermediate metabolite in the conversion of Hcy to Cys, does not require reduction or thiol derivatization, can be quantified concomitantly with the tHcy and tCys with the method disclosed herein. A high resolution mass spectrum of CysT is shown in FIG. 8. The data shows the CysT transition 223.1>134.04 can perform well as the quantitative transition in the described method. CysT and tCys can further increase the specificity of the assay by differentiating HCU from other rare diseases where Hcy is an elevated biomarker. Examples of such diseases include Cobalamin C-J, cystathioninuria, and remethylation disorders.

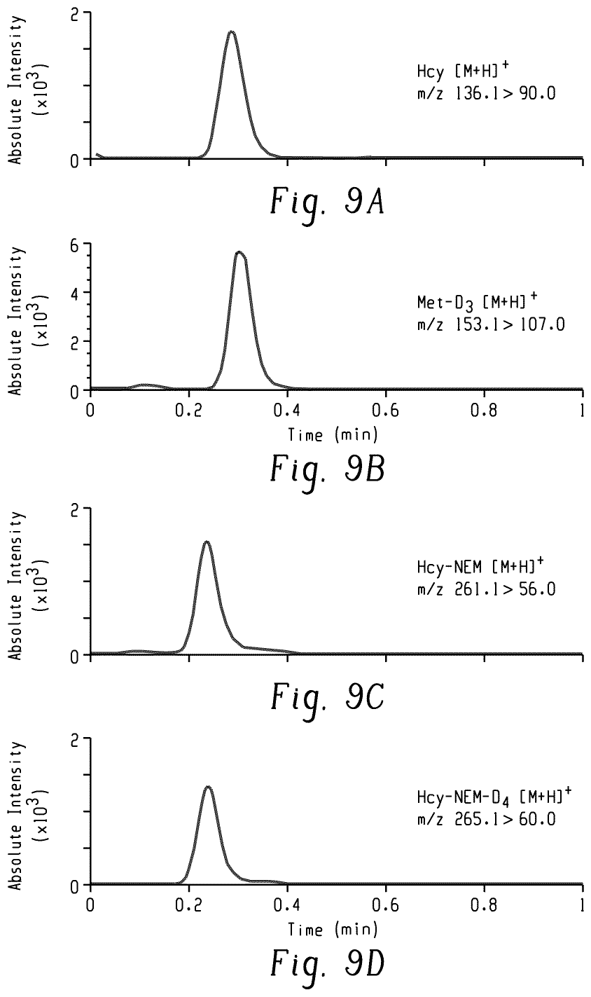

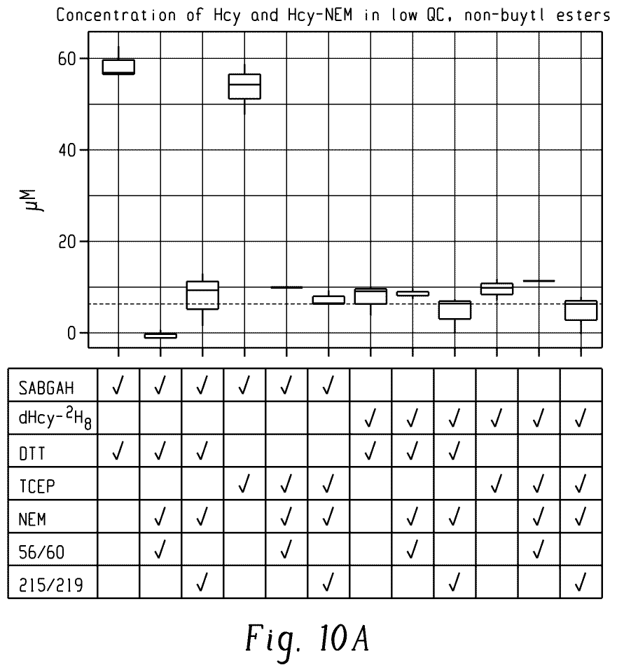

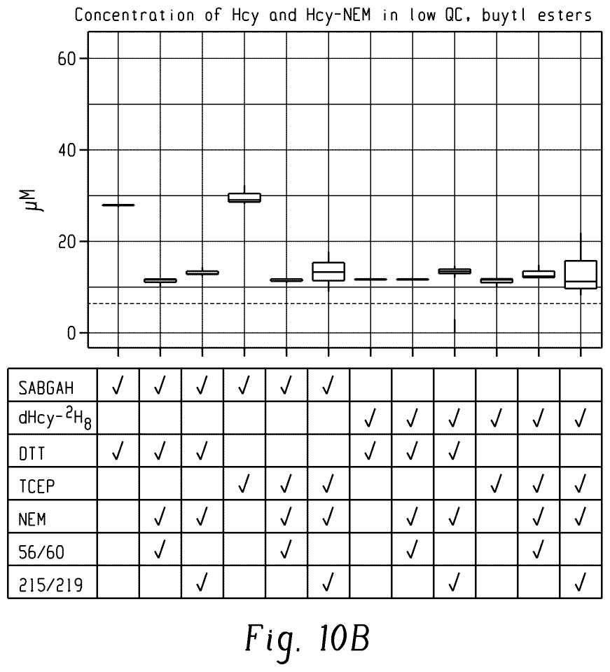

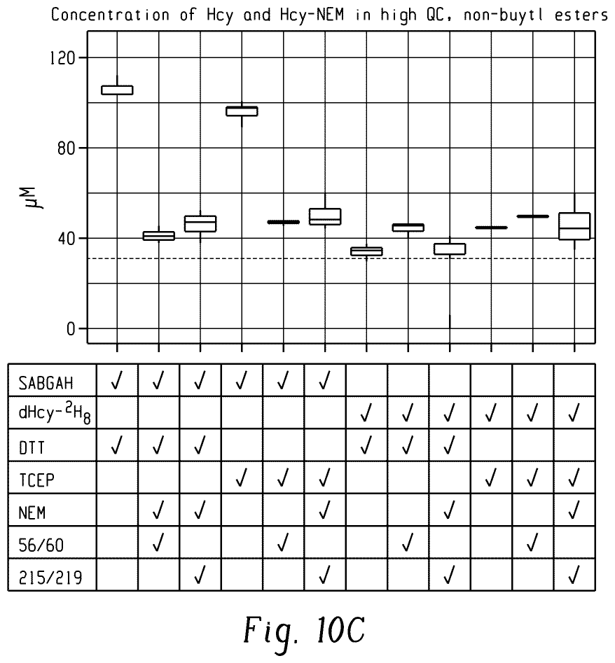

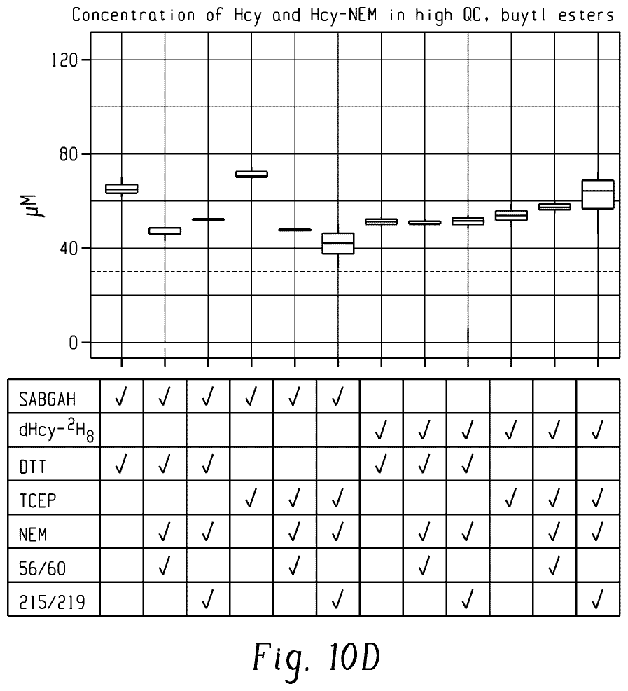

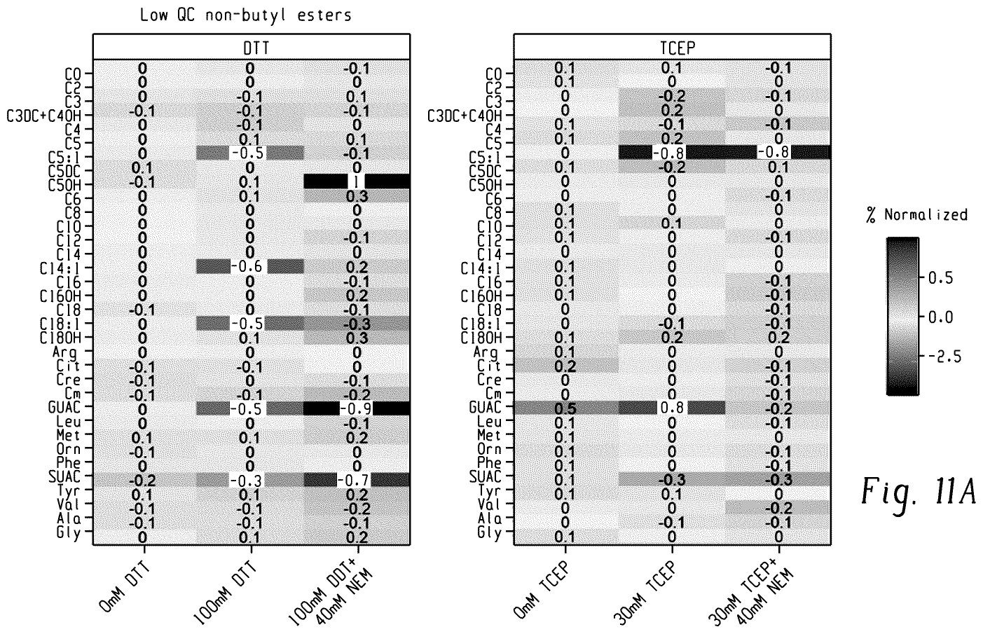

FIG. 9 compares the analysis of DBS extracts with or without TCEP and NEM treatment. Without TCEP and NEM treatment, the retention time of Hcy and Met-D3 are identical, thus differentiation between Hcy and the Met-D3 in-source fragment is not possible as shown in FIG. 9A and FIG. 9B. With TCEP and NEM treatment, NEM shifting the mass of reduced Hcy, making it possible to quantify Hcy-NEM without interference as shown in FIGS. 9C and 9D.

The methods described herein are efficient for detection of tHcy in first-tier high-throughput screening of blood samples such as DBS, without affecting other biomarkers, and provides significantly improved and reliable results. This could eliminate the need for second-tier screening and improve timelines. Thus, the methods can further comprise measuring an amino acid other than Hcy, an acylcarnitine, succinylacetone, adenosine, deoxyadenosine, guanidinoacetic acid, creatine, creatinine, and/or lysophospholipids simultaneously when a blood sample is analyzed for tHcy, tCys, or a combination thereof.

Elevated blood levels of Hcy (hyperhomocysteinemia) can represent a significant risk factor for various diseases or disorders such as cardiovascular diseases, neuropsychiatric illness, bone heath, and ectopia lentis, premature vascular and thrombotic disease. Hcy is also a biomarker for CBS deficiency or HCU.

Thus, the level of tHcy can be used to screen for CBS deficiency, HCU, or hypcrhomocysteinemia. The method comprises providing a blood sample from a subject, determining a tHcy level with a method as described herein, and if the tHcy in the blood sample is above a threshold, the subject matter may have CBS deficiency, HCU, or hyperhomocysteinemia.

As used herein, the subject can be a mammal such as a human, including without limitation a newborn human.

By “threshold” is meant a value selected to discriminate between subjects with and without CBS deficiency, HCU, or hyperhomocysteinemia. The threshold may be selected according to requirements, e.g., to identify subjects having a disease, or a particular increased risk thereof. In an embodiment, the threshold is an average tHcy concentration in the same sample type from a control population without CBS deficiency, HCU, or hyperhomocysteinemia.

Methods of multiplexing Hcy in a first-tier screening assay and methods of determining tHcy are further illustrated in the following examples.

### Examples

### Materials Used

Materials used include quality control materials (multi-level above and below average US cutoff for tHcy), proficiency test materials (presumptive normal and HCU+engineered samples), and linearity materials (multi-level materials to assess linearity of assay).

### Sample Preparation

DBS sample punches were placed into a plate of 96-wells and treated with 100 μl of a working internal standard solution (WISS) comprised of 80:20 acetonitrile:water containing 0.05% formic acid, with 0.0015% hydrazine hydrate, stable isotope-labeled standards for amino acids, acylcarnitines, succinylacetone, adenosine, deoxyadenosine, guanidinoacetic acid, creatine, creatinine, and dHcy-²H₈. WISS is also referred to as SABGAH herein. The treated DBS punches were then heated for 45 minutes at 45° C. TCEP was added, and the plate was sealed then shaken for five minutes. The supernatant was transferred to another 96-well plate, and dried.

NEM in either water or 50/50 methanol/water or 50/50 acetonitrile/water was added, and the plate was sealed then shaken for five minutes. The thiol derivatized samples were dried, and resuspended in 50/50 acetonitrile/water containing 0.1% formic acid.

Alternatively, NEM in either water, or 50/50 methanol/water by volume, or 50/50 acetonitrile/water by volume and the plate was sealed then shaken for five minutes. The thiol derivatized samples were analyzed without further drying and resuspension.

### Instrumentation and Data Analysis

All samples were analyzed via FIA on a Waters TQD MS/MS system and by direct infusion on a Thermo Q-Exactive Plus high-resolution mass spectrometer (HRMS). All data were analyzed using Mass Lynx, NeoLynx, and Xcalibur and Qual Browser. HRMS was used to assess and confirm the presence of interferences, and TQD was used to quantify Hcy, along with other biomarkers for method development and comparison.

### Results

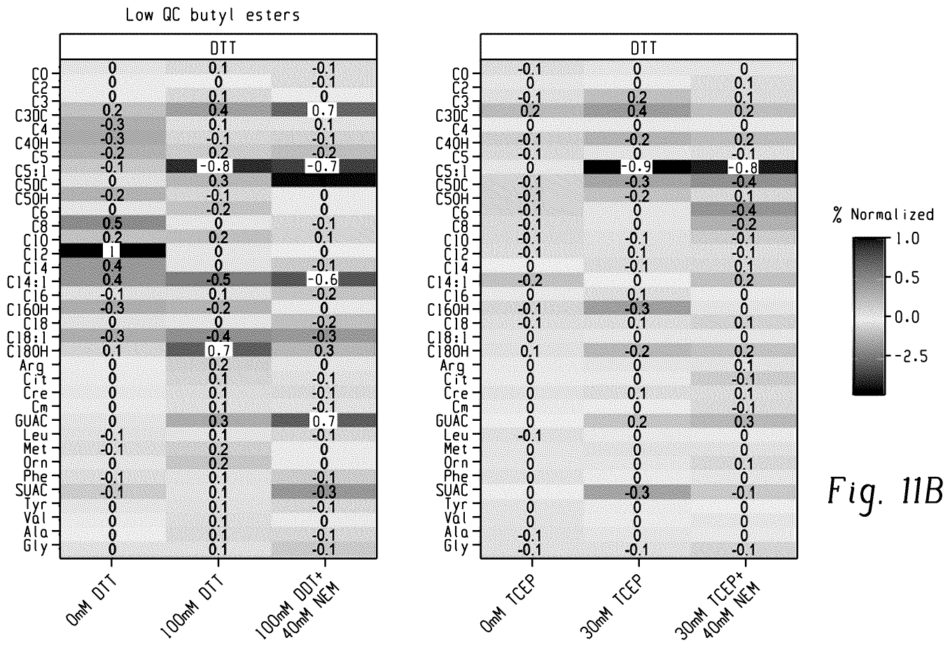

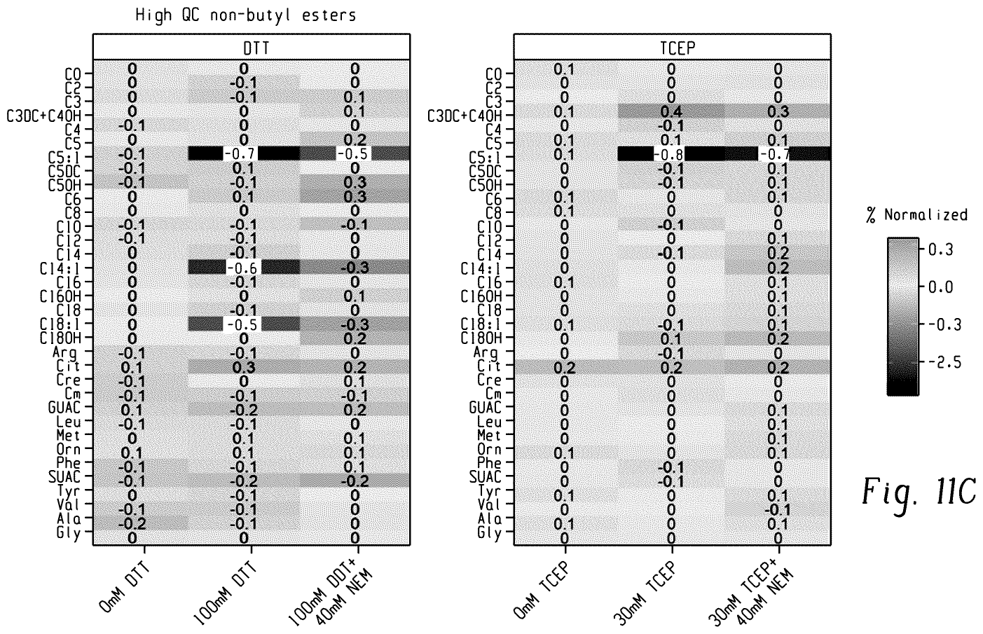

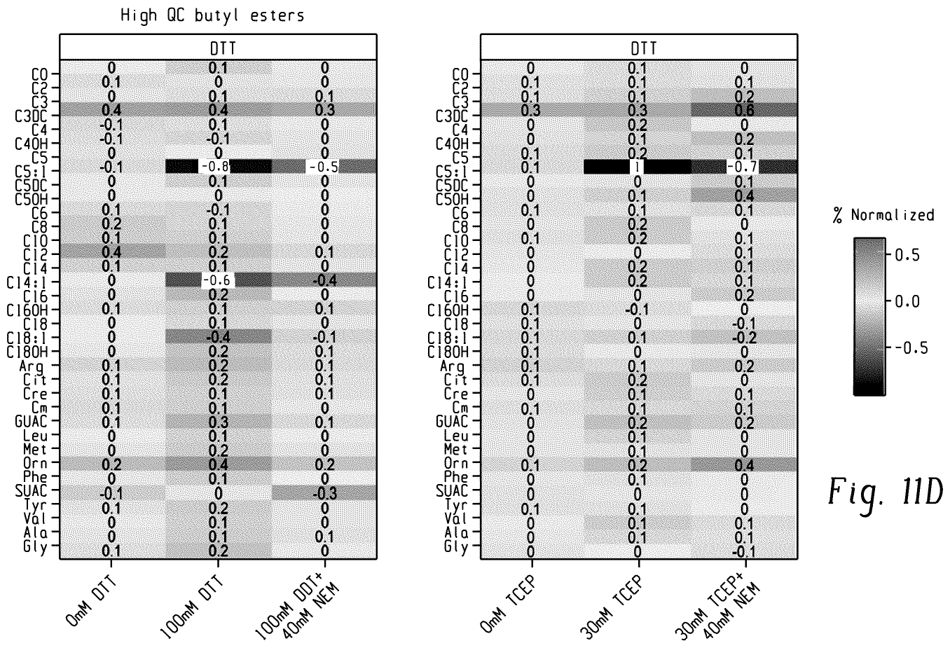

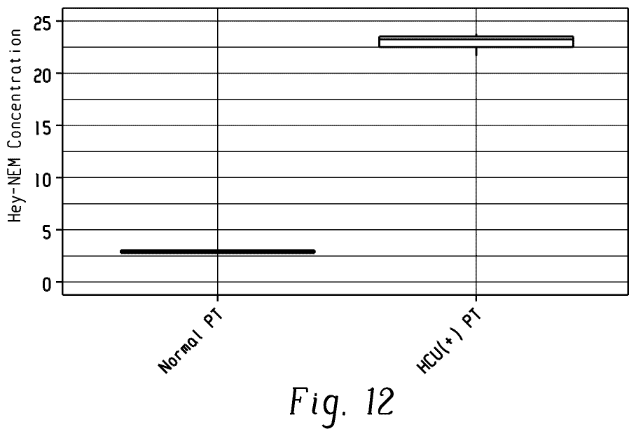

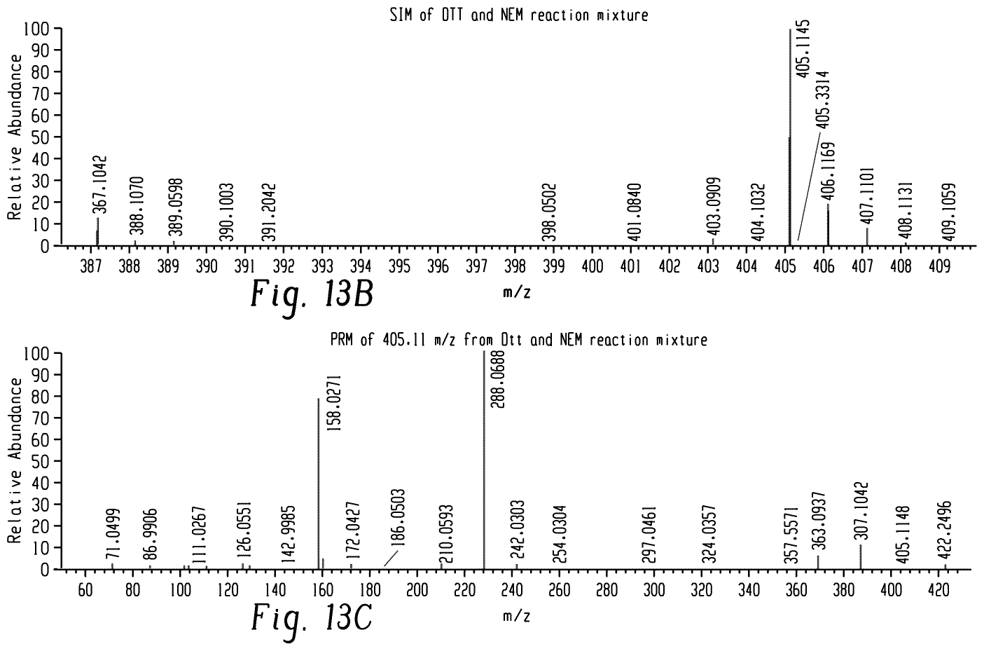

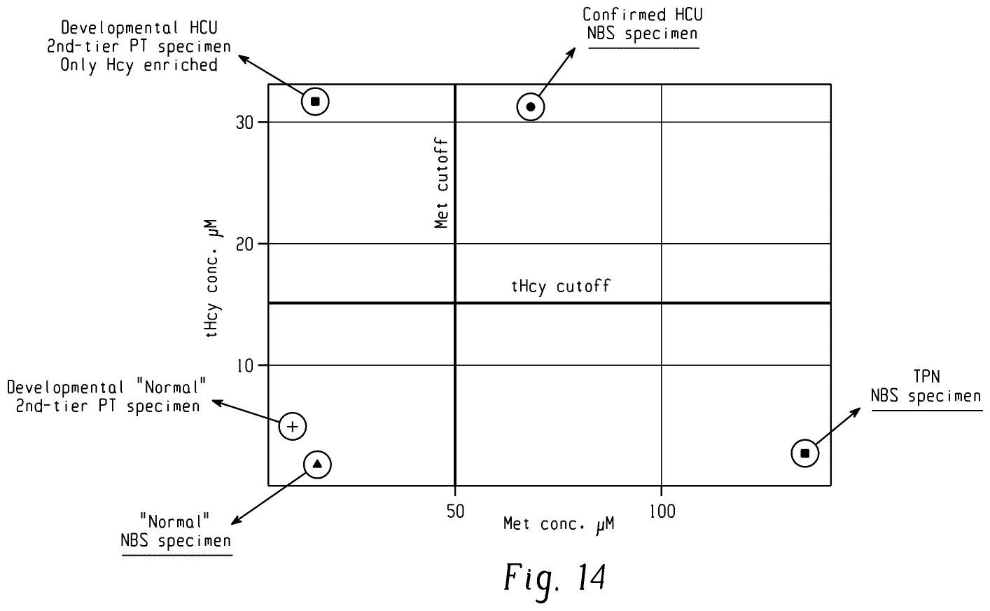

The Hcy-NEM reaction shown in FIG. 4 creates a new parent m/z and 2 characteristic product ion m/z (shown in FIG. 6). Utilization of reducing and NEM derivatizing agents successfully shifts the mass and mass-to-charge ratio of Hcy to remove interferences as seen in FIG. 10A-10D. Common newborn screening internal standards present in SABGH WISS were found to be interferants of Hcy, and treatment with reducing agents and NEM yielded tHcy concentrations similar to what was achieved by LC-MS/MS analysis of samples that were not treated with NEM (hashed line in FIGS. 10A-10D). Samples extracted with a WISS containing only dHcy-²H₈ (shown as dHcy-²H₈ in FIGS. 10A-10D) along with buffers described above (hydrazine and formic acid), yielded normal tHcy concentrations with and without the use of NEM. Both product ion pairs of unlabeled Hcy-NEM and labeled Hcy-2H4-NEM were assessed and the 56/60 pair tended to have less variability in samples with lower tHcy concentrations, indicating that 56/60 are an ideal quant pair with 215/219 better suited as qualifying product ions. FIGS. 11A-11D visualize the effects of adding pure water at reducing agent and thiol derivatizing steps of FIGS. 1-3, just the reducing agents, and the combination of reducing and thiol derivatizing agent, which are all normalized to controls; where the controls are identical samples processed without addition of pure water, reducing agents, and/or thiol derivatizing agents during sample prep. The heat map data are presented as percent normalized to control (referred to as Percent Normalized in FIGS. 11A-11D). FIGS. 11A and 11B display the effects of method modifications on low QCs processed through non-butyl ester and butyl ester derivatized sample prep. Both showed that samples treated with DTT and NEM had altered quantification due to the formation of DiNEM-DTT, which was a strong positive ion suppressing by-product (DiNEM-DTT). This impact was not seen in samples treated with TCEP and NEM. Biomarkers using surrogate internal standards including C3DC+C4OH, C3DC, C5:1, C10, C14:1, and C18:1 all showed altered quantified data compared to control due to differential ionization resulting from addition of reducing and thiol derivatizing agents. FIGS. 11C and 11D display effects of method modifications on high QC, which were consistent with findings from low QCs. FIG. 12 displays the method can distinguish presumptive normal and HCU positive proficiency test samples, analyzed in triplicate, that were treated with TCEP and NEM. FIG. 14 displays the results of developmental second-tier PTs engineered as presumptive normal and HCU positive, and clinical NBS specimens from newborns that were presumptive normal, administered trans parenteral nutrition (TPN), and confirmed HCU positive case. Samples were treated with TCEP and NEM to create Hcy-NEM for analysis of tHcy by FIA-MS/MS. FIG. 14 demonstrates the ability to distinguish HCU positive specimens from presumptive normal and TPN specimens using the developed method, increasing assay selectivity and sensitivity, while decreasing the false positive rate. FIG. 14 also demonstrates the method's clinical application in first-tier NBS sample, able to distinguish samples that are high tHcy in manufactured and clinical samples under FIA-MS/MS conditions. The results of linearity assay are shown in the table below.

| | | | 30 mM TCEP + 40 | | |
|---|---|---|---|---|---|
| | No TCEP or NEM | | mM NEM | | |
| Analyte | Slope | R\hat{ }2 | Slope | R\hat{ }2 | % Difference |
| Hcy-NEM | | | 0.66 | 0.99 | |
| GLY | 15.62 | 0.99 | 17.85 | 0.98 | 0.37% |
| ALA | 6.70 | 1.00 | 10.60 | 0.99 | 0.47% |
| VAL | 2.94 | 1.00 | 4.83 | 1.00 | 0.26% |
| LEU | 8.60 | 1.00 | 8.37 | 1.00 | −0.02% |
| MET | 3.32 | 0.99 | 2.69 | 1.00 | −0.02% |
| SUAC | 0.30 | 0.99 | 0.21 | 0.99 | −0.51% |
| PHE | 9.45 | 1.00 | 13.24 | 1.00 | 0.17% |
| ARG | 1.72 | 1.00 | 2.60 | 1.00 | 0.29% |
| CIT | 12.30 | 0.99 | 18.53 | 0.98 | 1.23% |
| TYR | 8.36 | 1.00 | 10.45 | 1.00 | 0.18% |
| CRN | 3.10 | 1.00 | 3.31 | 1.00 | 0.10% |
| GUAC | 0.36 | 0.97 | 0.34 | 0.97 | 0.02% |
| CRE | 7.03 | 1.00 | 9.45 | 0.99 | 0.34% |
| ORN | 3.06 | 1.00 | 5.54 | 0.99 | 0.62% |
| C0 | 2.84 | 1.00 | 3.58 | 1.00 | 0.15% |
| C2 | 1.80 | 1.00 | 2.38 | 0.99 | 0.23% |
| C3 | 0.20 | 1.00 | 0.47 | 0.99 | 0.79% |
| C4 | 0.05 | 1.00 | 0.07 | 0.99 | 0.38% |
| C5:1 | 0.03 | 0.99 | 0.02 | 0.99 | 0.08% |
| C5 | 0.12 | 0.99 | 0.13 | 0.99 | −0.01% |
| C3DC + C4OH | 0.02 | 0.99 | 0.03 | 0.99 | −0.02% |
| C6 | 0.07 | 0.99 | 0.08 | 0.99 | 0.01% |
| C5OH | 0.09 | 1.00 | 0.14 | 0.99 | 0.49% |
| C5DC | 0.09 | 0.99 | 0.10 | 0.99 | 0.15% |
| C8 | 0.22 | 1.00 | 0.30 | 0.99 | 0.27% |
| C10:2 | 0.07 | 0.99 | 0.09 | 0.97 | 2.05% |
| C10:1 | 0.09 | 0.99 | 0.19 | 0.86 | 14.19% |
| C10 | 0.09 | 0.99 | 0.08 | 0.99 | 0.02% |
| C12 | 0.09 | 1.00 | 0.20 | 0.99 | 1.08% |
| C14:1 | 0.04 | 1.00 | 0.10 | 0.96 | 3.54% |
| C14 | 0.07 | 1.00 | 0.12 | 1.00 | 0.28% |
| C16 | 0.16 | 1.00 | 0.18 | 1.00 | 0.11% |
| C16OH | 0.04 | 1.00 | 0.04 | 1.00 | 0.09% |
| C18:1 | 0.09 | 1.00 | 0.14 | 0.9 | 0.36% |
| C18 | 0.10 | 1.00 | 0.10 | 1.00 | 0.00% |
| C18OH | 0.02 | 1.00 | 0.03 | 1.00 | 0.00% |

The terms “a” and “an” do not denote a limitation of quantity, but rather denote the presence of at least one of the referenced item. The term “or” means “and/or.” The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on its scope unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this disclosure belongs.

1. A method of multiplexing homocysteine, cysteine, or a combination thereof in a first-tier screening assay, the method comprising:
contacting a blood sample with a reducing agent optionally in the presence of a solvent to convert at least one of a homocysteine dimer, a homocysteine-protein complex, a cysteine dimer different from the homocysteine dimer, or a cysteine-protein complex, in the blood sample to a homocysteine monomer, a cysteine monomer, or a combination thereof thereby forming a monomer sample;
reacting the homocysteine monomer, the cysteine monomer, or a combination thereof in the monomer sample with a thiol derivatizing agent to form a thiol derivatized sample comprising a thiol derivatized homocysteine monomer, a thiol derivatized cysteine monomer, or a combination thereof; and
optionally converting a carboxylic acid or a carboxylate group in the thiol derivatized homocysteine monomer, the thiol derivatized cysteine monomer, or a combination thereof to an ester.

2. The method of claim 1, wherein the thiol derivatizing agent is added to the blood sample before the monomer sample is formed.

3. The method of claim 1, wherein the monomer sample is formed by:
mixing the blood sample with the solvent, optionally at a temperature of 30° C. to 80° C., 35° C. to 70° C., or 40° C. to 50° C., to form an extracted sample; and
contacting the extracted sample with the reducing agent to form the monomer sample.

4. The method of claim 1, wherein the monomer sample is formed by heating the blood sample and the reducing agent in the presence of the solvent at a temperature of 30° C. to 80° C., 35° C. to 70° C., or 40° C. to 50° C.

5. The method of claim 1, wherein the solvent comprises an acid and at least one of acetonitrile, methanol, or water.

6. The method of claim 5, wherein a volume ratio of the acetonitrile relative to the water in the solvent is 95:5 to 20:80 or 90:10 to 60:40, and optionally the solvent comprises 0.01 to 0.5 volume percent or 0.01 to 0.1 volume percent of the acid based on a total volume of the solvent, and the acid comprises at least one of formic acid, acetic acid, trifluoroacetic acid, oxalic acid, or sulfosalicylic acid.

7. The method of claim 1, wherein
the monomer sample further comprises at least one of an internal standard for an amino acid other than homocysteine, cysteine, or a combination thereof, an internal standard for an acylcarnitine, an internal standard for succinylacetone, an internal standard for adenosine, an internal standard for deoxyadenosine, an internal standard for guanidinoacetic acid, an internal standard for creatine, an internal standard for creatinine, an internal standard for a lysophospholipid, an internal standard for homocysteine, or an internal standard for cysteine.

8. The method of claim 1, wherein the blood sample is a dried blood spot sample, a serum sample, or a plasma sample.

9. The method of claim 1, wherein the reducing agent comprises at least one of tris(2-carboxyethyl) phosphine or dithiothreitol.

10. The method of claim 1, wherein the reducing agent is introduced in a solid form or a liquid form, and optionally, the reducing agent is immobilized on a solid support.

11. The method of claim 1,
wherein the monomer sample comprises a solid precipitate and a supernatant; and the method further comprises:
separating the supernatant from the solid precipitate; and
optionally drying the supernatant.

12. The method of claim 1, wherein the thiol derivatizing agent comprises an N-alkyl maleimide.

13. The method of claim 1, wherein the reducing agent comprises tris(2-carboxyethyl) phosphine; and the thiol derivatizing agent comprises N-ethylmaleimide.

14. The method of claim 1, further comprising:
drying the thiol derivatized sample or the thiol-and-ester derivatized sample to form a dried derivatized sample; and
adding a second solvent to the dried derivatized sample to form a ready-to-use sample.

15. The method of claim 1 comprising:
heating the blood sample in the solvent to form the extracted sample, the solvent comprising an acid and at least one of acetonitrile, methanol, or water;
contacting the extracted sample with the reducing agent to convert the at least one of the homocysteine dimer, the homocysteine-protein complex, the cysteine dimer different from the homocysteine dimer, or the cysteine-protein complex in the extracted sample to the homocysteine monomer, the cysteine monomer, or a combination thereof forming the monomer sample;
separating a supernatant from a solid precipitate in the monomer sample;
optionally drying the supernatant to form a dried monomer sample;
combining the supernatant or the optionally dried monomer sample with an N-alkyl maleimide to form the thiol derivatized sample comprising the thiol derivatized homocysteine monomer, the thiol derivatized cysteine monomer, or a combination thereof; and
optionally converting the carboxylic acid or the carboxylate group in the thiol derivatized homocysteine monomer, the thiol derivatized cysteine monomer, or a combination thereof to the ester forming the thiol-and-ester derivatized sample.

16. The method of claim 15, wherein the supernatant or the optionally dried monomer sample is combined with the N-alkyl maleimide with at least one of water, methanol or acetonitrile.

17. A method of determining a level of total homocysteine, a level of total cysteine, or a combination thereof in a blood sample, the method comprising:
multiplexing homocysteine, cysteine, or a combination thereof in a first-tier screening assay according to the method of claim 1; and
analyzing the thiol derivatized sample, the thiol-and-ester derivatized sample, or the ready-to-use sample with mass spectrometry to quantify a level of total homocysteine, a level of total cysteine, or a combination thereof in the blood sample.

18. The method of claim 17, further comprising analyzing the thiol derivatized sample, the thiol-and-ester derivatized sample, or the ready-to-use sample with mass spectrometry to quantify a level of cystathionine in the blood sample.

19. The method of claim 17, wherein the thiol derivatized sample, the thiol-and-ester derivatized sample, or the ready-to-use sample is analyzed without prior chromatography separation.

20. The method of claim 17, wherein the thiol derivatized sample, the thiol-and-ester derivatized sample, or the ready-to-use sample is injected to a column prior to being analyzed with mass spectrometry, and wherein a total analysis time from injecting the sample to the column to quantifying the level of total homocysteine, the level of total cysteine, or a combination thereof, is 3 minutes or less.

21. The method of claim 17, wherein the thiol derivatized sample, the thiol-and-ester derivatized sample, or the ready-to-use sample is analyzed with tandem mass spectrometry.

22. A method of screening for cystathionine b-synthase deficiency, the method comprising:
providing a blood sample from a subject;
determining a level of total homocysteine in the blood sample according to the method of claim 17; and
determining that the subject has cystathionine b-synthase deficiency if the level of total homocysteine in the blood sample is above a threshold.

23. A method of screening for homocystinuria or hyperhomocysteinemia, the method comprising:
providing a blood sample from a subject;
determining a level of total homocysteine in the blood sample according to the method of claim 17; and
determining that the subject has homocystinuria or hyperhomocysteinemia if the level of total homocysteine in the blood sample is above a threshold.
